(19) United States
(12) Patent Application Publication
(10) Pub. No.: US 2026/0315333 A1
(43) Pub. Date: Oct. 15, 2026

(54) Advanced Bioactive Dental Cement for Bonding Ceramic Restorations

(71) Applicant: Burcin Calafiore, Wethersfield, CT (US)

(72) Inventor: Burcin Calafiore, Wethersfield, CT (US)

(21) Appl. No.: 19/177,452

(22) Filed: Apr. 11, 2025

(51) Int. Cl. A61K 6/30; A61K 6/17; A61K 6/75; A61K 6/76; A61K 6/77; A61K 6/818

(52) U.S. Cl. CPC A61K 6/30; A61K 6/17; A61K 6/75; A61K 6/76; A61K 6/77; A61K 6/818

(57) ABSTRACT

A dual-cure, bioactive resin cement is disclosed for bonding ceramic restorations (including veneers) to tooth structures. The cement integrates phosphate-based adhesive monomers (e.g., 10-MDP), bioactive glass, and nano-scale fillers (such as nano-hydroxyapatite or nano-zirconia) within a stress-relieving resin matrix. This combination enables strong chemical adhesion to both enamel/dentin and metal-oxide ceramics, while simultaneously releasing ions that foster remineralization and seal margins against microleakage. An optional rubberized or thio-urethane component reduces polymerization shrinkage stress, enhancing fracture toughness. By uniting universal bonding capability, bioactive ion release, and shock absorption in a single formulation, this invention addresses longstanding failures in veneer cementation—providing more durable bonds and allowing patients to bite confidently into harder foods without debonding.

### CROSS-REFERENCE TO RELATED APPLICATIONS

This application claims the benefit of, and priority to, U.S. Provisional Patent Application No. [63/700,625], filed on Sep. 28, 2024, entitled “Advanced Dental Cement for Bonding Porcelain Veneers”, the entire contents of which are incorporated by reference herein.

### FIELD OF THE INVENTION

This invention relates to dental materials, specifically resin-based, bioactive, dual-cure dental cements used for the adhesive bonding of ceramic restorations to tooth structures. More particularly, it concerns a dual-cure, bioactive cement formulation that integrates adhesive monomers, bioactive fillers, nanoparticles, and optional rubberized or thio-urethane modifiers to enhance both chemical bonding and long-term mechanical durability. The invention is applicable to various ceramic restorations, including (but not limited to) porcelain veneers, zirconia crowns, lithium disilicate inlays, and other prosthetic ceramics.

### 1. Technical Problem

Ceramic veneers are widely used in cosmetic dentistry to enhance tooth appearance. However, insufficient bonding reliability persists in many existing dental cements: patients frequently experience debonding (veneers coming loose) when biting into harder foods (e.g., apples, crusty bread). This leads to inconvenience, extra clinical visits, and diminished patient confidence.

### 2. Known Prior Art, Existing Products, and Their Limitations

### A. Commercially Available Resin Cements

A wide array of commercial cements address specific bonding challenges—ranging from self-adhesive convenience to bioactivity and MDP-based adhesion—but each provides only a partial solution:
1. Self-Adhesive Resin Cements
   Generally reduce clinical steps but tend to show lower bond strength on unetched enamel. Examples include the original 3M RelyX™ Unicem, which pioneered self-adhesive functionality using methacrylated phosphoric ester monomers and silanated fillers. While convenient, it lacks explicit bioactive features, such as nano-hydroxyapatite or 45S5 bio-glass, and thus does not maximize remineralization or shock absorption.
2. MDP-Containing Cements
   Ensure reliable chemical bonding to metal-oxide ceramics (e.g., zirconia). Products like Kuraray Panavia SA Cement Universal combine MDP and silane monomers for universal adhesion, but omit bioactive glass or nano-hydroxyapatite. Another example, Bisco TheraCem®, incorporates MDP plus calcium silicate fillers to achieve an alkaline pH, yet does not include nano-hydroxyapatite or a rubberized resin approach.
3. Bioactive or Ion-Releasing Cements
   Promise pH-neutralization or basic ion release, and can precipitate hydroxyapatite in marginal gaps. For instance, Parkell Predicta® Bioactive Cement releases calcium, phosphate, and fluoride while using MDP, but does not disclose nano-hydroxyapatite or 45S5 bio-glass as a filler.
   Pulpdent ACTIVA™ BioACTIVE Cement uses a “rubberized” resin plus bioactive glass for remineralization and reduced sensitivity. However, it omits 10-MDP, making it less suited for zirconia bonding without a separate primer.
4. Ivoclar Vivadent Variolink® Esthetic
   An aesthetic-driven adhesive resin cement requiring a full etch-and-bond protocol for best results. It offers strong bonds and good esthetics but lacks self-adhesive or bioactive capabilities.

In short, while certain products feature universal adhesion (via MDP), ion release (via bioactive fillers), or low-stress polymerization (through rubberized resins), no single widely available cement comprehensively merges all of these. This persistent gap—partially identified in prior filings—underscores the need for a more integrated solution that tackles universal bonding, remineralization, and stress relief within one formulation.

### B. Representative Patent Disclosures

Below are examples of patents describing partial innovations in resin cements:
1. US10180078465A1
   A self-adhesive, dual-cure resin cement that uses phosphate monomers (e.g., 10-MDP) plus basic fillers (e.g., calcium silicate). The pH shifts from acidic (~4) to basic (~8) post-cure, promoting apatite precipitation. No incorporation of nano-hydroxyapatite or pre-reacted 45S5 bio-glass is disclosed.
2. WO2018187375A1
   Claims a dual-cure system including 10-MDP, thioureas, and peroxides as initiators, with conventional fillers like silica. It lacks the specific combination of 45S5 bio-glass plus nano-hydroxyapatite.
3. EP1365727B1
   Introduces 45S5 bioactive glass into dental cements, enabling calcium/phosphate ion release and in-situ hydroxyapatite formation. However, it does not integrate phosphate monomers (MDP) or nano-fillers.
4. Pulpdent MCP Patent
   Describes a methacrylate-functionalized calcium phosphate in a rubberized resin matrix for remineralization, but does not include MDP for universal ceramic bonding, nor does it combine nano-HA or 45S5 glass.

### C. Conclusion & Novelty Positioning

Despite numerous commercial products and patent filings, no single known cement integrates:
45S5 bioactive glass for ion release and remineralization,
Nano-hydroxyapatite for interface reinforcement and nucleation,
10-MDP (or equivalent phosphate monomers) for universal chemical bonding, and
An optional rubberized or thio-urethane oligomer matrix for enhanced toughness and stress relief.

By uniting these features in a dual-cure resin system, the present invention addresses the shortfalls of prior art—offering a bioactive, stress-relieving, and universally adhesive cement that reliably bonds to tooth structure and ceramic restorations (including zirconia), all while promoting long-term remineralization and mechanical durability under normal or heavier bite forces.

### 3. Personal & Clinical Observations Demonstrating Market Need

From firsthand clinical experience and direct feedback, veneer detachment frequently occurs even under moderate biting forces. Existing solutions often impose technique-sensitive protocols (e.g., multiple primers or separate adhesives) or result in sub-optimal long-term bonds, particularly if partial dentin bonding is required.

Discussions with dental professionals indicate that repeated veneer failure commonly arises due to:
1. Poor Long-Term Bond at the tooth-cement interface, especially when the preparation includes dentin coverage.
2. Rigid or Brittle Cement that transmits masticatory forces directly to the veneer, risking fracture or debonding.
3. No Mechanism to remineralize micro-gaps or buffer acidic conditions at the margin, thereby increasing the likelihood of secondary caries or marginal breakdown.

Hence, there is a clear and urgent need for a universal, bioactive, low-stress resin cement that can reliably withstand the rigors of biting into tough foods without risking debonding or compromised bond integrity.

### SUMMARY OF THE INVENTION

The present invention provides a dual-cure, bioactive dental cement that addresses the persistent issue of veneer debonding by synergizing four core elements:
1. High Bond Strength
   Achieved via phosphate-based adhesive monomers (e.g., 10-MDP) for robust chemical adhesion to both tooth enamel/dentin and ceramic substrates, including metal-oxide ceramics such as zirconia and porcelain.
2. Remineralization & Margin Sealing
   Enabled by bioactive glass (e.g., 45S5) and nano-hydroxyapatite fillers, which release calcium/phosphate ions to foster hydroxyapatite formation at the tooth-cement interface. This seals microgaps, reduces microleakage, and bolsters long-term bond stability.
3. Stress Relief & Shock Absorption
   Provided through an optional low-stress resin component (e.g., thio-urethane or “rubberized” oligomers). This alleviates polymerization shrinkage stress, minimizes interfacial gaps, and absorbs occlusal forces, preventing veneer fracture or debonding.
4. Dual-Cure Convenience
   A dual-cure system ensures complete polymerization even where light penetration is limited by thicker or more opaque veneers. This prevents incomplete curing, discoloration, and compromised bond strength.

By integrating bioactive glass, nano-fillers, MDP-based monomers, and stress-relieving additives in a single cement, the invention strongly adheres to enamel, dentin, and a variety of ceramic surfaces. It also remineralizes micro-gaps, maintains high fracture toughness, and significantly reduces the risk of veneer detachment. Patients can thus bite into apples or similarly tough foods with confidence, free from the fear of debonding.

### DETAILED DESCRIPTION OF THE INVENTION

This section details the formulation and method of preparing and using the Advanced Bioactive Dental Cement in several embodiments, enabling a person skilled in the art to make and use the invention without undue experimentation.

### A. Composition of the Dental Cement

1. Resin Matrix
   Base Monomers:
   A combination of Bis-GMA, UDMA, TEGDMA, or similarly functional dimethacrylates forms the backbone of the resin. These monomers can be varied or combined to achieve the desired handling and mechanical properties.
   Dual-Cure Initiators:
   A photoinitiator (e.g., camphorquinone/amine) is included for light curing, paired with a chemical (self) cure system (e.g., benzoyl peroxide/amine). This ensures complete polymerization even in low-light or deeper regions.
   Optional Stress-Relieving Agents:
   Thio-urethane oligomers or “rubberized” resin segments—about 1-20 wt %—may be added to reduce polymerization shrinkage stress and enhance fracture toughness. These components mitigate stress at the tooth-cement interface, lowering the risk of debonding or veneer fracture.
2. Phosphate-Based Adhesive Monomers
   Examples: 10-MDP (10-methacryloyloxydecyl dihydrogen phosphate), GPDM, Phenyl-P, or functionally equivalent phosphoric acid esters.
   Concentration: Typically 2-10 wt % of the resin phase, enabling strong chemical bonding to both tooth structure (hydroxyapatite) and metal-oxide ceramics (e.g., zirconia). This universal adhesion feature is crucial for reliable veneer retention.
3. Bioactive Glass
   45S5-Type Bioglass (SiO₂—CaO—Na₂O—P₂O₅) or a similar composition, used at about 1-20 wt %.
   Mode of Action: Upon exposure to oral fluids, it releases calcium and phosphate ions, leading to in-situ formation of hydroxyapatite. This process helps seal margins and remineralize the tooth-cement interface, reducing microleakage and secondary carries risk.
4. Nano-Scale Fillers
   Nano-Hydroxyapatite (nHA): Typically 1-10 wt % to reinforce the bond with tooth structures, contribute to in-situ remineralization, and improve bond stability.
   Nano-Silica or Nano-Zirconia: Up to 15 wt % total, conferring mechanical reinforcement, wear resistance, and better polishability. Particle sizes are generally under ~100 nm for uniform dispersion and optimal handling.
5. Additional Additives
   Color/Translucency Adjusters (Aesthetic Modifiers): Ensure a natural look under porcelain veneers.
   Fluorescent Agents (Optional): Can mimic the natural fluorescence of teeth under various lighting conditions.
   Rheology/Flow Modifiers: For instance, fumed silica helps achieve a workable flow without sacrificing ideal film thickness. These additives fine-tune the cement's clinical handling and final appearance.

Collectively, these components create a dual-cure, bioactive resin cement capable of strong, durable bonding to enamel, dentin, and ceramic surfaces. The optional stress-relieving agents and bioactive fillers further bolster the mechanical integrity, shock absorption, and long-term stability required for veneer restorations under normal or heavier bite forces.

### B. Methods of Preparation

1. Mixing the Resin Matrix
   Combine Bis-GMA, UDMA, TEGDMA (or chosen monomers) at room temperature (~25 °C) under low-shear mixing.
   Introduce adhesive monomers (e.g., 10-MDP) into the blend, ensuring a homogeneous mixture.
2. Incorporating Nanoparticles
   Disperse nano-hydroxyapatite (nHA) or nano-silica/zirconia using high-shear mixing or ultrasonic agitation to prevent agglomeration.
3. Adding Bioactive Glass
   Gradually incorporate 45S5 or similar bioactive glass into the resin. Maintain continuous stirring to keep even particle distribution.
4. Optional Thio-Urethane/Rubberized Resin
   Add up to 20 wt % of a thio-urethane oligomer or rubber-like monomer to reduce shrinkage stress.
   Verify compatibility with the dual-cure system so that polymerization is not impeded.
5. Initiator & Final Adjustments
   Add photoinitiators (e.g., camphorquinone) and chemical initiators (e.g., benzoyl peroxide/amine) in appropriate ratios for dual-cure.
   Adjust viscosity/fillers for final clinical handling properties.
6. Quality Control
   Test for bond strength, compressive/flexural strength, degree of conversion, shelf stability, film thickness, and pH changes over time.

This integrated approach ensures comprehensive coverage of the mixing process, optional additives, and quality checks needed to produce a reliable bioactive, dual-cure dental cement suitable for bonding ceramic veneers.

### C. Methods of Application

1. Tooth Surface Preparation
   Clean the tooth surface thoroughly to remove debris.
   Etch enamel with phosphoric acid (e.g., ~35%) for about 15 seconds, then rinse and gently air-dry (avoiding overdrying dentin).
   Optional Universal Primer: If dentin is exposed, a universal primer may be beneficial, though the MDP monomer in this cement can partially self-etch if desired.
   Note: Self-adhesive function is possible if an added universal primer step is omitted, but a dedicated etch generally enhances bond strength to enamel.
2. Veneer Surface Preparation
   For Porcelain or Lithium Disilicate (silica-based ceramics): Apply the recommended hydrofluoric (HF) etch and silane coupling agent to optimize adhesion.
   For Zirconia (metal-oxide ceramic): Mild abrasion or direct bonding may suffice, as the MDP monomer in the cement chemically bonds to zirconia oxide.
3. Cement Application
   Apply a thin, uniform layer of cement on either the veneer or the tooth surface.
   Seat the veneer with firm yet gentle pressure, allowing any excess cement to flow out at the margins for easy removal.
4. Excess Removal & Curing
   Carefully remove cement overflows or marginal excess before final curing.
   Light-cure each accessible surface for ~20-30 seconds.
   In areas where light penetration is limited (e.g., thicker or more opaque veneers), self-cure or dual-cure properties ensure complete polymerization.
5. Finishing & Polishing
   Check occlusion and make necessary adjustments.
   Polish the margins to achieve a smooth transition between the veneer and tooth, reducing plaque retention and enhancing the long-term success of the restoration.

By following these steps, clinicians can maximize bond strength, minimize microleakage, and ensure durable retention of the veneer under normal or heavier bite forces.

### D. Exemplary Formulations

### Example 1A

Resin Base: 40 wt % Bis-GMA, 25 wt % UDMA, 5 wt % TEGDMA
Adhesive Monomer: 6 wt % 10-MDP
Bioactive Filler: 10 wt % 45S5 glass, 3 wt % nano-HA
Initiators: 1 wt % photoinitiators, 0.5 wt % chemical initiators
Optional: 5 wt % thio-urethane
Properties:
   Shear bond to etched enamel~25 MPa
   Compressive strength~200 MPa
   pH~7.5 after 30 min

### Example 1B

Resin Matrix: 40 wt % Bis-GMA, 30 wt % UDMA, 5 wt % TEGDMA, 5 wt % 10-MDP, 1 wt % photoinitiators
Bioactive Fillers: 8 wt % 45S5 glass, 2 wt % nano-HA
Optional: 5 wt % thio-urethane oligomer
Reported Results:
   Preliminary bond strength to etched enamel~25 MPa
   pH at 30 minutes~7.5

### Example 2A

Resin Base: 35 wt % Bis-GMA, 30 wt % TEGDMA, 2 wt % thio-urethane
Filler Package: 8 wt % nano-zirconia, 4 wt % 45S5 glass, 2 wt % nano-HA
Adhesive Monomer: 8 wt % GPDM
Suggested Use:
   Ideal for thicker zirconia veneers (opaque) requiring strong self-cure in deeper regions

### Example 2B

Resin Matrix: 50 wt % Bis-GMA, 25 wt % TEGDMA, 8 wt % MDP
Filler Package: 10 wt % nano-zirconia+5 wt % 45S5, 2 wt % photoinitiators/accelerators
Performance:
   Designed primarily for high-stress veneer applications (e.g., molar region)
   Testing indicates compressive strength~200 MPa and good color stability over 3 months in a simulated environment

Note: These examples illustrate how different component ratios or monomer selections can tailor properties (e.g., compressive strength, bond durability, or color stability) for various clinical scenarios. Each formulation demonstrates the bioactive, dual-cure nature of the invention, reliably bonding to tooth structure and ceramic surfaces while optionally incorporating stress-relieving or nano-reinforcing additives.

### Independent Claims

1. A dual-cure, bioactive dental cement composition for bonding a ceramic restoration (including veneers) to a tooth surface, comprising:
   (a) a resin matrix capable of both light and chemical curing,
   (b) at least one phosphate-based adhesive monomer selected from the group consisting of 10-MDP, GPDM, Phenyl-P, or functionally equivalent monomers,
   (c) a bioactive glass filler in an amount sufficient to promote remineralization at the tooth-cement interface, and
   (d) nanoparticulate fillers selected from nano-hydroxyapatite, nano-silica, nano-zirconia, or any combination thereof,
   wherein the composition is configured to form a durable bond with both tooth structure (enamel/dentin) and ceramic surfaces.
2. The dental cement composition of claim 1, wherein the bioactive glass comprises a 45S5-type composition in about 1-20 wt % of the total cement.
3. The dental cement composition of claim 1, wherein the phosphate-based adhesive monomer comprises 10-methacryloyloxydecyl dihydrogen phosphate (10-MDP) in a concentration of about 2-10 wt % of the resin matrix.
4. The dental cement composition of claim 1, wherein the nanoparticulate fillers include nano-hydroxyapatite in a range of about 1-10 wt % to enhance bond durability and remineralization.
5. The dental cement composition of claim 1, further comprising a stress-relieving resin additive, selected from thio-urethane oligomers or rubberized resin, in an amount of about 1-20 wt %, thereby reducing polymerization shrinkage stress and improving fracture toughness.
6. A method of bonding a ceramic veneer to a tooth surface using the composition of claim 1, comprising the steps of:
   (a) preparing the tooth surface by cleaning and optionally etching,
   (b) applying the dental cement to either the tooth or the veneer surface,
   (c) seating the veneer with light or gentle pressure,
   (d) removing excess cement from the margins, and
   (e) curing the cement via light activation and/or self-curing,
   wherein the phosphate-based monomer chemically bonds to both tooth structure and ceramic.
7. A kit for bonding a ceramic restoration to a tooth surface, comprising:
   (a) an acid etchant or primer solution,
   (b) the dental cement composition of claim 1 provided in one or more containers, and
   (c) instructions for mixing and applying said dental cement,
   wherein the bioactive glass and phosphate-based monomer within said cement cooperate to promote remineralization and durable adhesion to both tooth structure and ceramic surfaces.

### Information Disclosure

Applicant will submit an Information Disclosure Statement (IDS) listing all known relevant prior art, including but not limited to: existing commercial resin cements with ion release or MDP, relevant research articles, and any patents discovered in prior searches. This ensures transparency and assists the examination process.

### Conclusion

The present invention addresses the long-standing challenge of veneer detachment by synergizing bioactive fillers, nano-scale reinforcement, universal adhesive chemistry (e.g., phosphate-based monomers like 10-MDP), and optional low-stress resin components. By enabling strong, remineralizing, and shock-absorbing bonds to tooth and ceramic surfaces, this dual-cure cement significantly reduces the risk of veneer failure—allowing patients to bite confidently into apples (and more) without fear of debonding.

In summary, the Advanced Bioactive Dental Cement described herein:
Universal: Adheres securely to multiple substrates (enamel, dentin, zirconia, porcelain).
Bioactive: Releases ions that foster hydroxyapatite formation and help seal margins.
Shock-Resistant: Incorporates stress-relieving or rubberized resin plus nano-fillers to keep veneers intact under high masticatory forces.
Clinically Convenient: A dual-cure formula that sets reliably, regardless of light penetration or veneer thickness.

Consequently, this invention fills a critical gap in modern dentistry by providing a cement that unites universal bonding capability, inherent remineralization, and stress-relief—thereby reducing clinical failures and enhancing patient comfort.

Having thus described several embodiments, it will be apparent to those skilled in the art that changes and modifications may be made without departing from the spirit or scope of the appended claims.

1. A dual-cure, bioactive dental cement composition for bonding a ceramic restoration (including veneers) to a tooth surface, comprising:
(a) a resin matrix capable of both light and chemical curing,
(b) at least one phosphate-based adhesive monomer selected from the group consisting of 10-MDP, GPDM, Phenyl-P, or functionally equivalent monomers,
(c) a bioactive glass filler in an amount sufficient to promote remineralization at the tooth-cement interface, and
(d) nanoparticulate fillers selected from nano-hydroxyapatite, nano-silica, nano-zirconia, or any combination thereof,
wherein the composition is configured to form a durable bond with both tooth structure (enamel/dentin) and ceramic surfaces.

2. The dental cement composition of claim 1, wherein the bioactive glass comprises a 45S5-type composition in about 1-20 wt % of the total cement.

3. The dental cement composition of claim 1, wherein the phosphate-based adhesive monomer comprises 10-methacryloyloxydecyl dihydrogen phosphate (10-MDP) in a concentration of about 2-10 wt % of the resin matrix.

4. The dental cement composition of claim 1, wherein the nanoparticulate fillers include nano-hydroxyapatite in a range of about 1-10 wt % to enhance bond durability and remineralization.

5. The dental cement composition of claim 1, further comprising a stress-relieving resin additive, selected from thio-urethane oligomers or rubberized resin, in an amount of about 1-20 wt %, thereby reducing polymerization shrinkage stress and improving fracture toughness.

6. A method of bonding a ceramic veneer to a tooth surface using the composition of claim 1, comprising the steps of:
(a) preparing the tooth surface by cleaning and optionally etching,
(b) applying the dental cement to either the tooth or the veneer surface,
(c) seating the veneer with light or gentle pressure,
(d) removing excess cement from the margins, and
(e) curing the cement via light activation and/or self-curing,
wherein the phosphate-based monomer chemically bonds to both tooth structure and ceramic.

7. A kit for bonding a ceramic restoration to a tooth surface, comprising:
(a) an acid etchant or primer solution,
(b) the dental cement composition of claim 1 provided in one or more containers, and
(c) instructions for mixing and applying said dental cement,
wherein the bioactive glass and phosphate-based monomer within said cement cooperate to promote remineralization and durable adhesion to both tooth structure and ceramic surfaces.
